(19) United States
(12) Patent Application Publication
(10) Pub. No.: US 2016/0184596 A1
(43) Pub. Date: Jun. 30, 2016

(54) MINIMALLY INVASIVE IMPLANTABLE BRAIN STIMULATION DEVICES AND METHODS FOR IMPLANTING SAME

(71) Applicant: THE REGENTS OF THE UNIVERSITY OF CALIFORNIA, Oakland, CA (US)

(72) Inventors: Itzhak FRIED, Los Angeles, CA (US); Ilan OREN-BEN, Herzliya Pituach (IL); Tamir WOLF, Herzliya Pituach (IL)

(21) Appl. No.: 14/910,644

(22) PCT Filed: Aug. 5, 2014
(86) PCT No.: PCT/US14/49829

§ 371 Date: Feb. 5, 2016

Related U.S. Application Data
(60) Provisional application No. 61/862,274, filed on Aug. 5, 2013.

(51) Int. Cl. A61N 1/378; A61N 1/375; A61N 1/372; A61N 1/05; A61N 1/36

(52) U.S. Cl. CPC A61N 1/3787; A61N 1/0534; A61N 1/0539; A61N 1/3605; A61N 1/37229; A61N 1/3756

(57) ABSTRACT

A brain stimulation device includes: a threaded head portion; a needle portion connected to the threaded head portion; at least one wireless power source arranged in proximity to the threaded head portion, the at least one wireless power source being configured to generate electrical energy for the brain stimulation device responsive to a wireless power signal being received by the brain stimulation device; and at least one electrode arranged at least partially on an outside surface of the needle portion in electrical communication with the at least one wireless power source, wherein the at least one electrode is configured to transmit an electrical signal responsive to receiving the electrical energy from the at least one wireless power source. A method of using the brain stimulation device is also described.

### CROSS-REFERENCES TO RELATED APPLICATIONS

This application claims priority to U.S. Provisional Patent Application No. 61/862,274 filed Aug. 5, 2013 and entitled MINIMALLY INVASIVE IMPLANTABLE BRAIN STIMULATION DEVICES AND METHODS FOR IMPLANTING SAME, the entire contents of which is incorporated herein for all purposes by this reference

### BACKGROUND

Deep brain stimulation (DBS) devices may be implanted in the head of a patient to treat various neurological conditions, such as Parkinson's disease, depression, and epilepsy. In general, DBS devices include electrodes that are implanted through a surgical burr hole drilled through the top of the skull of the patient and fed down through the brain until they reach a target site. The electrodes provide electrical stimulation for the target site and, as such, require a connection to a power supply that is situated nearby or is carried around by the patient and physically connected to the DBS device.

Example target sites for DBS electrodes commonly include thalamic nuclei (for example, Ventralis Intermediate Nucleus), the subthalamic nucleus or the globus pallidus, which are each located deep within the brain. As such, the DBS electrodes are guided through the top regions of the brain until the electrodes reach the site of interest within the brain. In certain applications, the final location may be set based on physiology information. Surgical techniques for implanting existing DBS electrodes often require invasive procedures that may have negative consequences for patients, such as increased risks of infection and neurological complications including weakness, speech and swallowing difficulties, and abnormal sensations or cognitive deficits. Furthermore, apparatus and methods currently used for implanting DBS devices often require long procedures and general anesthesia which is normally required for placement of the neurostimulator units and tunneling of connecting cables. Procedures that are less invasive may reduce the likelihood that negative consequences may occur and may improve recovery times and the overall patient experience. Accordingly, a brain stimulation device that may be implanted through a minimally invasive procedure would benefit the treatment of neurological conditions requiring brain stimulation.

### SUMMARY

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

In one aspect, a brain stimulation device may include a threaded head portion, a needle portion connected to the threaded head portion, at least one wireless power source arranged in proximity to the threaded head portion and configured to generate electrical energy for the brain stimulation device responsive to a wireless power signal being received by the brain stimulation device, and at least one electrode arranged at least partially on an outside surface of the needle portion in electrical communication with the at least one wireless power source and configured to transmit an electrical signal responsive to receiving the electrical energy from the at least one wireless power source. The brain stimulation device may be implanted into the skull of an animal in order to stimulate the brain of the animal through a minimally invasive procedure. A human is a non-limiting example of an animal. Once implanted, the brain stimulation device may be powered and may communicate with one or more external devices through wireless means.

In another aspect, a minimally invasive method of implanting a brain stimulation device into the skull of an animal may include forming a hole in the skull of the animal, placing a brain stimulation device comprising a threaded head portion and a needle portion connected to the threaded head portion and having at least one electrode arranged at least partially on an outside surface thereof into the hole such that the at least one electrode contacts a target site of a brain located within the skull and the threaded head portion contacts an external perimeter of the hole, and affixing the brain stimulation device to the skull by rotating the brain stimulation device such that threads of the threaded head portion screw the brain stimulation device into the hole until the threaded head portion is substantially flush with an external surface of the skull.

### BRIEF DESCRIPTION OF THE DRAWINGS

FIGS. 1A and 1B depict an illustrative implantable brain stimulation device implanted within a patient according to some embodiments.

FIG. 2 depicts an illustrative brain stimulation device implanted in the skull of a patient according to an embodiment.

FIG. 3 depicts an illustrative brain stimulation device according to an embodiment.

FIGS. 4A and 4B depict an illustrative brain stimulation device according to an embodiment.

### DETAILED DESCRIPTION

The terminology used in the description is for the purpose of describing the particular versions or embodiments only, and is not intended to limit the scope.

The technology described herein relates generally to devices configured to provide bioelectrical current to (to “stimulate”) and to receive bioelectrical current from (to “sense”) portions of an animal brain, including, without limitation, the entorhinal region of the human brain and/or the hippocampus. The entorhinal region as used herein may generally include the entorhinal cortex and/or the perforant path. In particular, embodiments provide a brain stimulating device that may be implanted in the skull of an animal flush or substantially flush therewith. In an embodiment, the brain stimulating device may have a minimal profile in order to allow for implantation into a patient through various minimally invasive procedures. The brain stimulating device may be configured as a therapeutic system for treating a neurological condition.

The brain stimulating device may include at least a head portion and a needle portion. The head portion may include a threaded portion (for instance, a threaded screw-like portion) configured to anchor the brain stimulating device to the skull of a patient. The needle portion may extend from the head portion with at least a portion thereof in contact with at least a portion of the animal brain. At least one lumen may be arranged within the needle portion, for instance, that is configured to enable drug delivery to the portion of the animal brain adjacent to the needle portion. The same or another lumen may be configured to receive a guide wire that facilitates the insertion of an electrode to a target site in the brain, for example, by providing a mechanical balance between rigidity and flexibility by the needle portion. The guide wire may be removed after the electrode is in place.

Various electronics (electronic components or electronic elements) may be located adjacent to the head portion on and/or within the brain stimulating device to perform various functions for the device. Certain electronics may also be arranged on and/or within the needle portion, such as electrodes, contacts, sensors, or the like. As used herein, the term “electronics” may refer generally to any electrical device, element, circuit, printed circuit board (PCB), system-on-a-chip (SoC), integrated circuit, electrical coil, chip (for example, a microchip), application-specific integrated circuit (ASIC), resistor, capacitor, inductor, diode, transistor, receiver, transmitter, transducer, electrode, electrical contact, electrical lead, antenna, radio frequency element, batteries, miniature batteries, or the like. For example, the electronics may include, without limitation, a wireless receiver, a wireless transmitter, a wireless inductor, an antenna, and electrodes. In an embodiment, the electronics may be configured to generate a bioelectrical signal configured to stimulate at least a portion of an animal brain. In an embodiment, the wireless electronics may be configured as “near-field” communication elements, such as near-field wireless transmitter, receiver, and/or power harvesting elements. In another embodiment, the electronics may be configured to receive one or more bioelectrical signals from one or more portions of an animal brain.

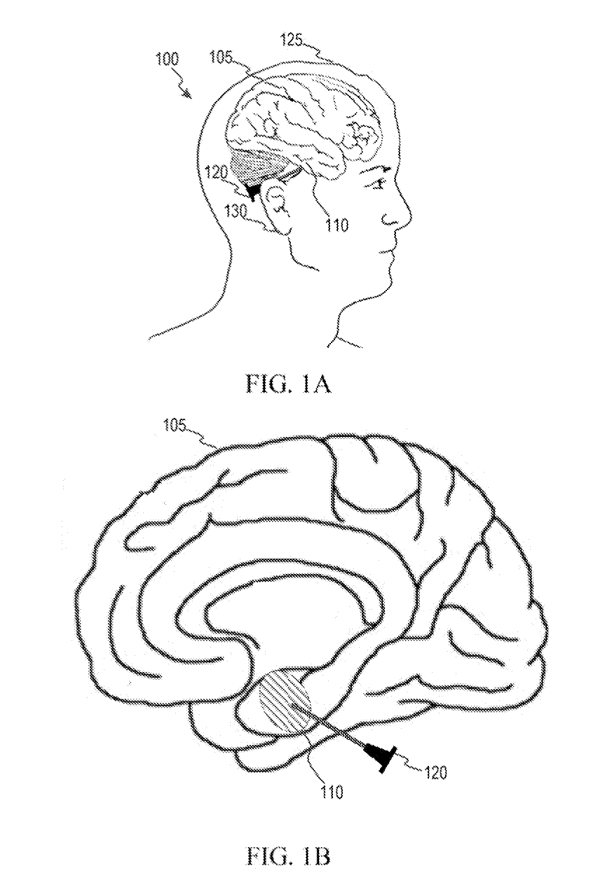

FIGS. 1A and 1B depict an illustrative implantable brain stimulation device implanted within a patient according to some embodiments. As shown in FIGS. 1A and 1B, a brain stimulation device 120 may be implanted within the brain 105 of a patient 100. According to some embodiments, the brain stimulation device 120 may be configured to have a portion thereof positioned within the entorhinal region 110 of the brain. For example, the brain stimulation device 120 may be implanted through the skull of the patient 100 at a position located near the ear 130 thereof. Implantation of the brain stimulation device 120 in immediate proximity to the ear, such as above the root of the zygoma of the patient 100, may provide a relatively straight trajectory from the insertion point to the entorhinal region 110 of about 2 centimeters to about 5 centimeters.

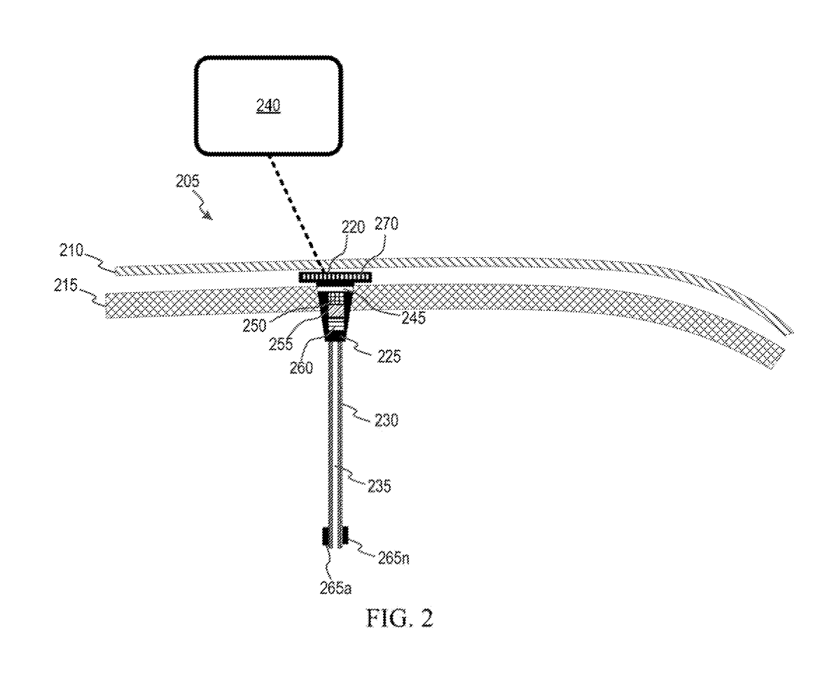

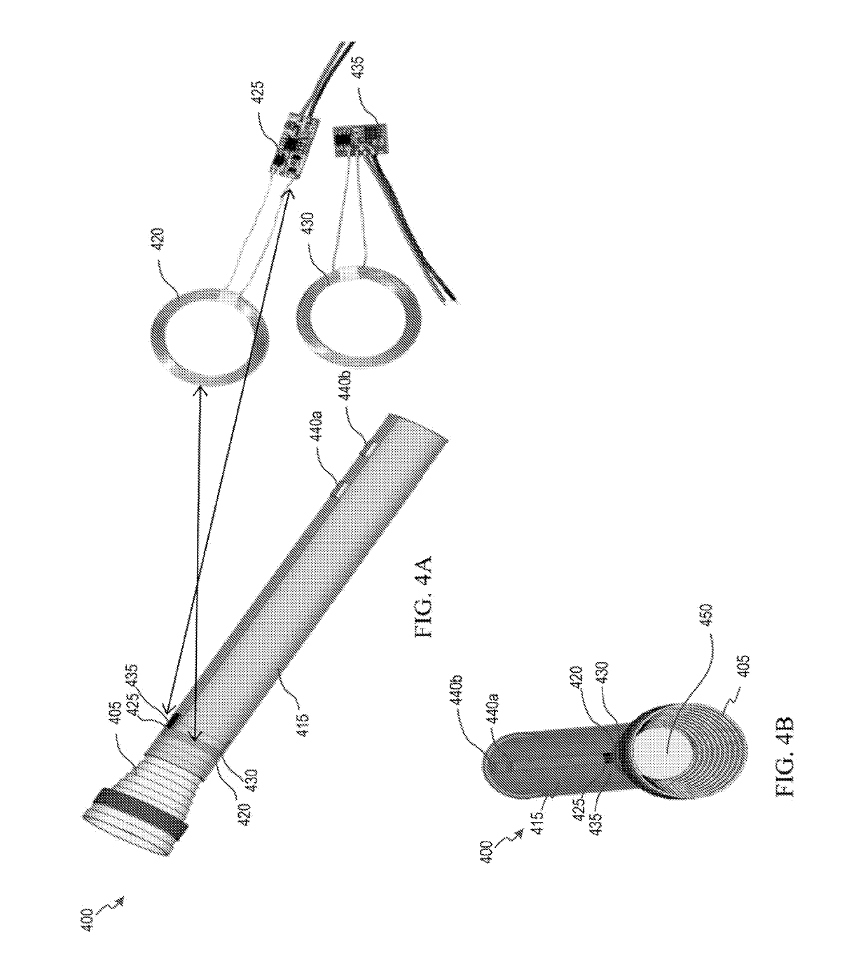

In an embodiment, a method for implantation of the device may include making an incision in the temporalis muscle and implanting the brain stimulation device 120 within a small hole cut in the skull near the zygomatic arch, for example, a twist drill hole. However, embodiments are not so limited as any incision and/or implantation zone capable of operating according to some embodiments is contemplated herein. In general, the site of insertion may be selected based on the type of disorder being treated, patient symptoms and physical characteristics, targeted brain areas, or the like. In particular, the site of insertion may be selected to position the antenna of the brain stimulation device (see FIGS. 2, 4A and 4B, below) in proximity to the ear of the patient. According to some embodiments, positioning the brain stimulation device in proximity to the ear of the patient may provide, among other things, a convenient solution for patients to carry an external power source (see, for example, device 240 of FIG. 2) in a similar manner to devices for hearing aid, such as a behind-the-ear (BTE) device.

Although FIGS. 1A and 1B depict the brain stimulation device 120 as being positioned within the entorhinal region 110 of the brain 105, embodiments are not so limited as any region of the brain that may be stimulated by and/or transmit bioelectrical signals to the brain stimulation device according to some embodiments is contemplated herein. Non-limiting examples of brain regions that may be used with the brain stimulation device 120 include the amygdala, the hippocampus, the parahippocampus, the perirhinal cortex, the subthalamic nucleus, the globus pallidus interna, thalamic nuclei such as the lateral ventromedial thalamic nucleus (VMI) or the anterior nuclear (AN) group, the periaqueductal gray, the periventricular gray, the internal capsule, the ventral posterolateral nucleus, the ventral posteromedial nucleus, or the like. Stimulation of the entorhinal region has been demonstrated to provide multiple medical benefits. Non-limiting examples of medical benefits includes increased memory, such as increased spatial memory, and the ability to provide “on-demand” stimulation during live learning by the patient.

The brain stimulation device 120 may be inserted into a portion of the brain 105 and/or configured once implanted in the brain to treat and/or receive information associated with various neurological disorders, conditions, behaviors, functions, activities, or the like. Illustrative and non-restrictive neurological disorders, conditions, behaviors, activities and/or functions include Alzheimer's disease, amyotrophic lateral sclerosis (ALS), Parkinson's disease, Huntington's Disease, Guillain-Bane syndrome, myasthenia gravis, chronic idiopathic demyelinating disease (CID), amelioration of neurological activity, psychological activity, conscious state activity, thought activity, depression, apathy, amnesia, Kluver-Bucy syndrome, mania, obsessive compulsive disorder, limbic epilepsy, rage, psychosis, social disdecorum, anxiety/panic disorders, attention or cognitive disorders, autistic spectrum disorders, mood disorders, bipolar disorder, dysthymic disorder, multiple sclerosis, dyskinesia, tremor, dystonia, chorea and ballism, tic syndromes, Tourette's Syndrome, myoclonus, drug-induced movement disorders, Wilson's Disease, Paroxysmal Dyskinesias, Stiff Man Syndrome, Akinetic-Ridgid Syndromes and Parkinsonism, epilepsy, tinnitus, pain, phantom pain, diabetes neuropathy, and headaches.

FIG. 2 depicts an illustrative brain stimulation device implanted in the skull of a patient according to an embodiment. As shown in FIG. 2, a brain stimulation device 220 may be implanted in the skull 215 of a patient 205. The brain stimulation device 220 may include a head portion 225 and a needle portion 230. In an embodiment, at least a portion of the outer surface of the head portion 225 may be tapered and may include a threaded portion configured to anchor the brain stimulation device 220 to the surface of the skull 215, for instance, in a manner similar to a metal screw being anchored in wood or other similar material. During the insertion procedure, a hole may be drilled into the skull 215 (for example, a surgical burr hole) and the brain stimulation device 220 inserted through the hole until the threaded portion contacts the perimeter of the hole. The brain stimulation device 220 may then be rotated (or “screwed”) into the hole until the head portion 225 is securely fastened to the skull. According to some embodiments, the form of the head portion 225 and/or the size and shape of the hole may be configured such that the head portion is flush or is substantially flush with the outer surface of the skull 215. Incisions made during the procedure for implanting the brain stimulation device 220 will heal in a manner such that a layer of skin and/or muscle 210 may cover the head portion 225.

The brain stimulation device 220 may be inserted using various medical procedures including, without limitation, minimally invasive procedures, stereotactic procedures, and/or the like. Due to the minimal profile of the brain stimulation device 220, the insertion procedure may be performed in a relatively short time and with relatively minor invasiveness when compared with the procedures required for inserting and/or implanting conventional devices. For example, insertion of the brain stimulation device 220 does not require guiding cannulas, o-rings, and/or physical (for example, non-wireless) electrical connections.

A lumen 235 may be arranged within the needle portion 230. The lumen 235 may extend through the head portion 225 such that the lumen may be accessed therefrom. At least a portion of the needle portion 230 may be flexible, substantially flexible, semi-rigid, rigid, or any combination thereof. For instance, at least a portion of the needle portion 230 may exhibit flexibility so that the needle portion may flex and/or move as the brain moves within the skull of the patient. In an embodiment, a rigid structure may be at least partially arranged within the lumen 235 during the implantation procedure to provide sufficient rigidity for the brain stimulation device 220 to be inserted into the brain. After the brain stimulation device 220 has been inserted, the rigid structure may be removed. A non-limiting example of a rigid structure is a guide wire.

In an embodiment, the lumen 235 may be used for the delivery of materials in and/or around the target site of the brain stimulation device 220 after implantation. Illustrative materials include drugs, contrast agents and other pharmaceuticals. The lumen 235 may be accessed through the head portion 225, for instance, through a needle inserted through a top portion of the head portion. Accordingly, the top portion of the head portion 225 may be formed from a material capable of being punctured by a needle or other similar element to allow a needle to move through the lumen 235 to deliver a material in and/or around a target site within the brain. According to some embodiments, the lumen 235 may be in fluid communication with a material delivery device (not shown) configured to deliver a material through the lumen and to a target site in the brain. In an embodiment, the material delivery device may include an infusion pump or other similar automatic infusion device. In an embodiment, the material delivery device may include a material delivery system configured to be worn by the patient, such as a behind-the-ear (BTE) device, and deliver the material through the lumen 230 to the target site. Non-restrictive examples of infusion pumps include peristaltic pumps, osmotic pumps, accumulator-type pumps, drive-spring diaphragm pumps, or the like.

The head portion 225 and the needle portion 230 may be formed from and/or coated with various materials, including various biocompatible materials. Non-limiting examples of such materials include ceramics, metals, metal alloys, polymers, silicone, carbides, alloys, polytetrafluoroethylene, polyp-xylylene) polymers (including vapor deposited poly(p-xylylene) polymers), aluminum oxide, calcium oxide, any combination thereof, or the like.

The brain stimulation device 220 may be associated with various electronic elements that perform various functions including, without limitation, communication, transmitting electrical signals, receiving electrical signals, conditioning electrical signals, power generation, and power harvesting. For example, one or more electrodes 265a-n (“contacts,” “leads” or “micro-leads”) may be positioned at or near the tip of the needle portion 230. The electrodes 265a-n may be formed from various materials known to those having ordinary skill in the art, including, without limitation, platinum, palladium, silver, titanium, iridium, copper, silver oxide, gold, alloys thereof, and/or any combination thereof. The electrodes 265a-n may be in electrical communication with a power source 245 configured to supply a voltage to the electrodes 265a-n sufficient to generate a signal within the target site capable of operating according to the requirements for treating a neurological condition. For example, each electrode 265a-n may be configured to provide an electrical signal having various electrical pulse characteristics, such as particular voltages, pulse widths, and frequencies. In an embodiment, the brain stimulation device 220 may be configured to provide a voltage of about 0.5 volts to about 10 volts to each of the electrodes 265a-n. According to some embodiments, the brain stimulation device 220 may be configured to provide “on-demand” stimulation during learning to facilitate neurological treatment for certain conditions.

According to some embodiments, the brain stimulation device 220 may include a plurality of electrodes 265a-n, for example, arranged around the outer surface of the needle portion 230. In an embodiment, the plurality of electrodes 265a-n may be arranged at or substantially at the tip of the needle portion 230. Each of the plurality of electrodes 265a-n may be individually activated so that a physician may select different positions around the needle portion 230 for stimulating the target site and/or for receiving signals from the surrounding brain area. According to various embodiments, the plurality of electrodes 265a-n may include 2 electrodes, 3 electrodes, 4 electrodes, 5 electrodes, 10 electrodes, as many as one hundred or more electrodes, and values or ranges between any of the above values (including endpoints). According to some embodiments, certain of the plurality of many electrodes 265a-n may be configured for stimulation while other electrodes may be configured for sensing. In an embodiment, at least a portion of the plurality of electrodes 265a-n may be configured for both stimulation and sensing.

The power supply 245 may include various elements and/or systems configured for wireless power generation and/or wireless power harvesting through one or more methods. For example, the power supply 245 may include a coil, a circuit, a receiver, energy harvesting elements, an antenna, energy storage elements, a capacitor, a battery, a miniature battery, any combination thereof, or the like configured to generate power responsive to receiving radio frequency (RF) signals, being subjected to an electromagnetic field, or being exposed to other wireless power signals known to those having ordinary skill in the art. The signals and/or field may be generated by an external device 240. In an embodiment, the external device 240 may be configured as a miniature stimulator for transferring power to the power supply 245. In an embodiment, the external device 240 may be configured as a BTE device that may be worn by the patient. For example, the external device 240 may be a BTE device that is worn by the patient during the day and charged at night. In an embodiment, the external device 240 may be configured as a device to be carried by the patient in a location that may power and/or communicate with more than one implanted brain stimulation device 220. For example, the external device 240 may be configured to be carried in proximity to the patient, such as in a shirt pocket or around the neck of the patient, to power and/or communicate with more than one brain stimulation device 220, for instance, implanted on different sides of the head of the patient.

The power supply 245 may be configured to generate power using various processes, such as induction, transduction, or the like. For instance, the power supply 245 may induce a voltage when subjected to an electromagnetic field. In another instance, the power supply 245 may induce a voltage responsive to receiving a wireless signal, such as an RF signal.

According to some embodiments, the power supply 245 may operate through at least one of radio charging, inductive charging and resonance charging.

According to some embodiments, the energy harvesting elements of the power supply 245, such as an antenna, may be positioned on or within the brain stimulation device 220 such that they are external to the skull. In this manner, the signals, such as RF signals or an electromagnetic field, do not have to penetrate or deeply penetrate into the skull. According to some embodiments, the brain stimulation device 220 may be configured to include electrical and thermal conductance characteristics to dissipate energy, such as excess energy for operating the brain stimulation device, outside of the brain. For example, the brain stimulation device 220 may be configured to dissipate energy to the skull and/or an outer layer of the skull.

The power supply 245 may include multiple components configured to perform various functions. For example, the power supply 245 may include an antenna for receiving signals, such as RF signals or other wireless signals, and a converter for converting the signals to electrical current. For instance, an RF power supply 245 may include an RF-to-direct current (DC) converter. One or more conditioning elements may be configured to condition the current for use by the electrodes 265a-n and/or other electronic components. Illustrative examples of RF power supplies/energy harvesters are the Powerharvester® family of wireless power components configured to convert wireless RF energy to DC energy sold by the Powercast Corporation of Pittsburgh, Pa. In another example, the power supply 245 may include a coil configured to induce a voltage responsive to being subjected to an electromagnetic field.

According to some embodiments, the brain stimulation device 220 may include multiple power supplies 245 that are, for instance, configured to provide power to one or more of the electronic components positioned in or within the brain stimulation device. The electronic components may be selected used, and/or configured such that they have a low energy profile/consumption. In this manner, the brain stimulation device 220 may operate with low individual and collective energy requirements suitable for use with a wireless power supply 245.

According to some embodiments, one or more electronic elements, may be arranged in and/or around a top region 270 of the head portion 225. Electronic elements in and/or around the top region 270 may be removed, replaced, exchanged, or otherwise accessed with minimally invasive procedures. For example, a battery may be placed in the top region 270 and may be replaced by a surgeon making an incision in the thin layer of skin and/or muscle above the implantation site of the brain stimulation device 220 to access the battery (for instance, by removing a cover or other similar enclosing element). Various of the electronic elements, such as any of those described in association with the wireless power supply 245, described according to some embodiments may be arranged in and/or around the top region 270 for access by a surgeon through a minimally invasive procedure.

The brain stimulating device 220 may include a stimulation control element 250 configured to control stimulation through the electrodes 265a-n and/or other electronic components. According to various embodiments, the stimulation control element 250 may include a circuit, integrated circuit, PCB, SoC, or the like. The stimulation control element 250 may include various sub-elements configured to provide signal processing, signal filtering, and signal control functions for the brain stimulating device 220. In an embodiment, the stimulation control element 250 may use local field potential (LFP) analysis to implement stimulation control. According to various embodiments, the stimulation control element 250 may use one or more of the following to implement stimulation control: a low-noise amplifier (LNA), an analog/digital converter circuit (ADC) (for example, a log-ADC), a digital log-filter, a digital signal processor (DSP) (for example, a log-DSP) current stimulator, a clock generator, a proportional-integral (PI) controller, a proportional-integral-derivative (PID) controller.

A wireless transmitter 255 may be arranged on or within the brain stimulation device 220, such as, on an outside surface of the head portion 225. The transmitter 255 may be configured to transmit signals to the external device 240 using wireless transmission protocols known to those having ordinary skill in the art, including RF signals. According to some embodiments, the external device 240 may be configured as any type of electronic and/or logic device configured to communicate with the brain stimulation device 220. Non-limiting examples of external devices 240 include a personal computer (PC), a laptop, a smart phone, a tablet computing device, a BTE device, a power transfer device, an RF transmitter, an electromagnetic field generator, and any other electronic and/or computing devices now known or developed in the future. According to some embodiments, the external device 240 may include multiple devices. For example, one external device 240 may be a BTE device for charging the brain stimulation device 220. Another external device 240 may be a logic device for receiving data from the brain stimulation device 220. Yet another external device 240 may be a control electronic device configured to wirelessly control the stimulation of the target site through the electrodes 240a-n. Additional and/or alternate external devices 240 may be used within the scope of this disclosure.

The external device 240 may be configured to receive and/or process the signals received from the transmitter 255. For example, the external device 240 may receive neural signals produced at a target site, received by one of the electrodes 265a-n and transmitted to the transmitter 255 for communication to the external device. The external device 240 may process the signals and/or provide them to a physician as information about the neurological condition of the patient. In an embodiment, at least a portion of the electrodes 265a-n may be configured to sense the theta wave, for example, by using at least two electrodes to detect the impact of stimulation on the target site. The large amplitude/low frequency (hippocampal) theta rhythm may indicate, among other things, active memory circuitry. In some embodiments, the external device 240 may be configured to record various information to assess neurological activity, such as electroencephalography (EEG) information. According to some embodiments, the acquired neurological signals, such as the theta rhythm or other EEG parameters, may be used to control stimulation.

The brain stimulating device 220 may include a receiver 260 configured to receive signals from the external device 240. The receiver 260 may be configured to receive signals from the external device 240 using wireless transmission protocols known to those having ordinary skill in the art. For example, the receiver 260 may be configured to receive RF signals from the external device 240. In an embodiment, the signals may be configured to control the brain stimulating device 220 by configuring the operation of the electrodes 265a-n and/or the stimulation control element 250, directing the electrodes to stimulate the target site, or the like. In an embodiment, the receiver 260 may be an energy harvesting component of a power supply 245 that is configured to receive RF signals.

According to some embodiments, the electronic components, such as the power supply 245, the stimulation control element 250, the transmitter 255, and the receiver 260, may be positioned on the head portion 225 in order to facilitate heat dissipation away from the inside of the skull and toward the outside of the skull.

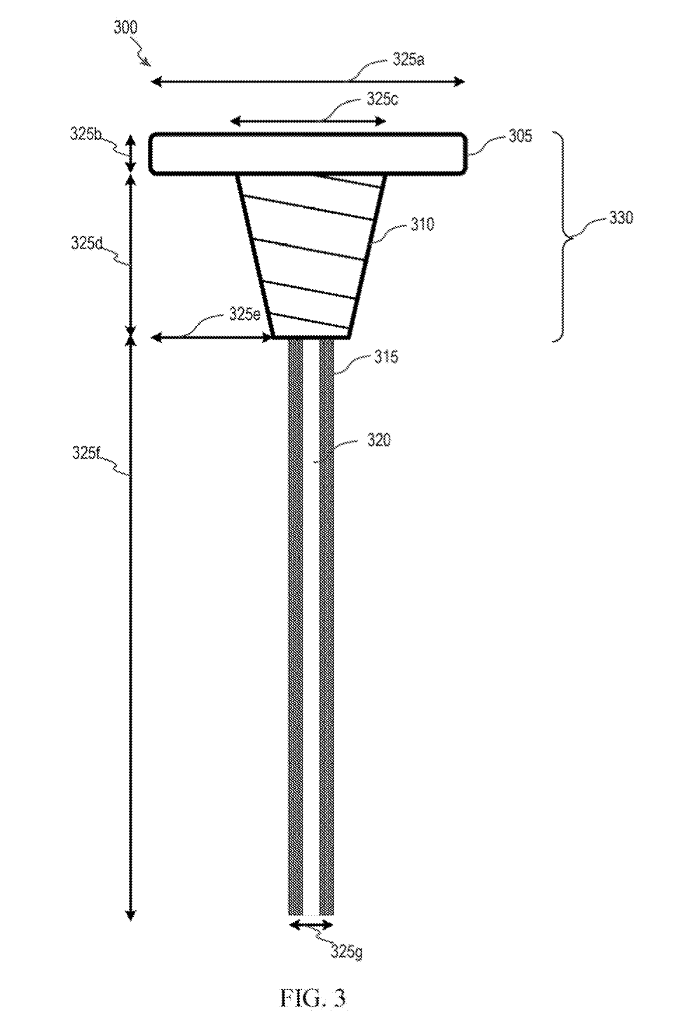

FIG. 3 depicts an illustrative brain stimulation device according to an embodiment. As shown in FIG. 3, a brain stimulation device 300 may include various portions, such as a needle portion 315 and a head portion 330. The head portion 330 may include various portions, such as a top portion 305 and a threaded portion 310. In an embodiment, the top portion 305 and the threaded portion 310 may be separate elements. In another embodiment, the top portion 305 and the threaded portion 310 may be formed as a continuous piece. The top portion 305 or a portion thereof may be removable from the brain stimulation device 300 and/or configured to be opened to expose a lumen 320 extending through the needle portion 315. According to some embodiments, a needle may access the lumen 320 by penetrating the top portion 305, for example, to deliver materials to a target site adjacent to the tip of the needle portion 315. The threaded portion 310 may include threads configured to facilitate the rotation or “screwing” of the threaded portion into a hole in the skull of a patient and to affix the brain stimulation device 300 thereto, in a manner similar to a screw being rotated within wood or other similar material.

The head portion 330, portions 305, 310 thereof, and/or the needle portion 315 may be flexible, semi-flexible, rigid, semi-rigid, or any combination thereof. In an embodiment, the head portion 330 may be rigid or semi-rigid and the needle portion 315 may be flexible. In an embodiment, the head portion 330 and the needle portion 315 may be formed as separate pieces. In another embodiment, the head portion 330 and the needle portion 315 may be formed as a continuous piece.

The dimensions 325a-325g of the brain stimulation device 300 may be configured to provide a minimum profile and to allow for implantation through minimally invasive procedures and may be influenced by the targeted brain structure, neurological condition, patient characteristics, or other similar factors. A top portion width 325a may be about 5 millimeters (mm), about 7 mm, about 10 mm, about 12 mm, about 15 mm, and values or ranges between any two of these values (including endpoints). A top portion height 325b may be about 0.25 mm, about 0.5 mm, about 0.75 mm, about 1 mm, about 2 mm, and values or ranges between any two of these values (including endpoints). A threaded portion top width 325c may be about 1 mm, about 2 mm, about 3 mm, about 4 mm, about 5 mm and values or ranges between any two of these values (including endpoints). A threaded portion height 325d may be about 1 mm, about 2 mm, about 3 mm, about 4 mm, about 5 mm, about 7.5 mm, about 10 mm, and values or ranges between any two of these values (including endpoints). In an embodiment, the threaded portion height 325e may be about less than about 6 mm when the entorhinal region is the target region and may be about 6 mm or greater (for instance, about 10 mm) when another region is the target region. A distance 325e from a threaded portion bottom to an end of the top portion may be about 0.25 mm, about 0.5 mm, about 0.75 mm, about 1 mm, about 2 mm, about 3 mm, about 4 mm and values or ranges between any two of these values (including endpoints).

The needle portion 315 may have a length 325f if about 0.5 mm, about 1 mm, about 5 mm, about 10 mm, about 15 mm, about 20 mm, about 50 mm, about 100 mm, about 125 mm and values or ranges between any two of these values (including endpoints). The needle portion 315 may have a width 325g of about 0.25 mm, about 0.5 mm, about 0.75 mm, about 1 mm, about 2 mm, about 3 mm, and values or ranges between any two of these values (including endpoints).

FIGS. 4A and 4B depict an illustrative brain stimulation device according to an embodiment. FIG. 4A depicts a side view of a brain stimulation device 400 having a threaded head portion 405 and an elongated needle portion 415. A pair of electrodes 440a, 440b may be arranged on an outside surface of the needle portion 415. A transmitter 425 in electrical communication with a coil 420 (for instance, a coil of copper wire) and a receiver 435 in electrical communication with a coil 430 may be arranged on an outer surface of the brain stimulation device 400. For example, the transmitter 425 and the receiver 435 may be located at a region where the head portion 405 joins with the needle portion 415. FIG. 4B depicts a view of the brain stimulation device 400 at the head portion 405 end showing the lumen 450 extending through the needle portion 415 and through the head portion. In an embodiment, the head portion 405 may be capped after implantation of the brain stimulation device 400 in the patient.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to”). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example), the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “ a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “ a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, or the like. As a non-limiting example, each range discussed herein can be readily broken down into a lower third, a middle third, and an upper third. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A brain stimulation device comprising:
a threaded head portion;
a needle portion connected to the threaded head portion;
at least one wireless power source arranged in proximity to the threaded head portion, the at least one wireless power source being configured to generate electrical energy for the brain stimulation device responsive to a wireless power signal being received by the brain stimulation device; and
at least one electrode arranged at least partially on an outside surface of the needle portion in electrical communication with the at least one wireless power source, wherein the at least one electrode is configured to transmit an electrical signal responsive to receiving the electrical energy from the at least one wireless power source.

2. The brain stimulation device of claim 1, wherein the brain stimulation device is configured to stimulate and/or to receive neural signals from an entorhinal region and/or hippocampus of an animal brain.

3. The brain stimulation device of claim 1, wherein the threaded head portion comprises at least one of a ceramic material, a metal material, a metal alloy material, and a polymer.

4. The brain stimulation device of claim 1, wherein the threaded head portion comprises at least one of a transmitter and a receiver.

5. The brain stimulation device of claim 4, wherein the threaded head portion comprises at least one coil in electrical communication with at least one of the transmitter and a receiver.

6. The brain stimulation device of claim 1, wherein at least a portion of the needle portion is flexible.

7. The brain stimulation device of claim 1, further comprising a lumen arranged within the needle portion and extending through at least a portion of the threaded head section.

8. The brain stimulation device of claim 7, wherein the lumen is configured as a material delivery device for a target area of an animal brain.

9. The brain stimulation device of claim 8, wherein the brain stimulation device is configured to receive a material from an external pump device.

10. The brain stimulation device of claim 1, wherein the at least one wireless power source comprises an RF harvesting device.

11. The brain stimulation device of claim 1, wherein the at least one wireless power source is configured to generate the electrical energy responsive to being subjected to a magnetic field.

12. The brain stimulation device of claim 1, further comprising a guide wire arranged at least partially within the lumen, the guide wire being configured to provide rigidity to the needle portion.

13. A minimally invasive method of implanting a brain stimulation device into the skull of an animal, the method comprising:
forming a hole in the skull of the animal;
placing the brain stimulation device comprising a threaded head portion and a needle portion connected to the threaded head portion and having at least one electrode arranged at least partially on an outside surface thereof into the hole such that the needle portion contacts a target site of a brain located within the skull and the threaded head portion contacts an external perimeter of the hole;
affixing the brain stimulation device to the skull by rotating the brain stimulation device such that threads of the threaded head portion screw the brain stimulation device into the hole until the threaded head portion is substantially flush with an external surface of the skull

14. The method of claim 13, further comprising charging the brain stimulation device by transferring a wireless power signal from an external device to at least one wireless power source arranged in proximity to the threaded head portion, the at least one wireless power source being configured to generate electrical energy for the brain stimulation device responsive to the wireless power signal being received by the brain stimulation device.

15. The method of claim 13, wherein the target site comprises an entorhinal region of an animal brain.

16. The method of claim 13, further comprising:
arranging a guide wire at least partially within the lumen to provide rigidity to the needle portion when placing the brain stimulation into the hole; and
removing the guide wire through the head portion subsequent to affixing the brain stimulation device to the skull.
